(19) United States
(12) Patent Application Publication
(10) Pub. No.: US 2026/0364142 A1
(43) Pub. Date: Nov. 19, 2026

(54) METHOD FOR ATTENUATING DERMATITIS

(71) Applicant: ADVAGENE BIOPHARMA CO., LTD., Taipei City (TW)

(72) Inventors: Yu-Shen HSU, Taipei City (TW); Hau-Chiun WU, Taipei City (TW); Ming-I CHANG, Taipei City (TW); Yi-Shiuh HWANG, Taipei City (TW)

(73) Assignee: ADVAGENE BIOPHARMA CO., LTD., Taipei City (TW)

(21) Appl. No.: 19/680,038

(22) Filed: May 18, 2026

Related U.S. Application Data
(60) Provisional application No. 63/808,015, filed on May 19, 2025.

(51) Int. Cl. A61K 38/16; A61K 9/00; A61K 9/06; A61K 47/38; A61P 17/06

(52) U.S. Cl. CPC A61K 38/164; A61K 9/0014; A61K 9/06; A61K 47/38; A61P 17/06

(57) ABSTRACT

The present disclosure provides a method for treating a subject having dermatitis, such as atopic dermatitis, eczema or psoriasis, comprising administering to the subject a therapeutically effective amount of a composition comprising detoxified Escherichia coli labile toxin (LT), such as LTh(αK), via a topical route.

### FIELD OF THE INVENTION

This invention pertains to a method for alleviating inflammatory skin conditions. Specifically, the invention relates to a method for treating dermatitis, including, but not limited to, eczema, psoriasis (PSO), and atopic dermatitis. The method involves the topical application of a composition containing a genetically detoxified Escherichia coli heat-labile toxin (LT), particularly LTh(αK), in a cellulose-based thickener to address chronic inflammatory epidermal syndromes.

### BACKGROUND OF THE INVENTION

The epidermis, the skin's outermost layer, is primarily composed of specialized cells called keratinocytes. These cells form the skin's first line of defense, creating a protective barrier against extracellular infections, harmful substances, and dehydration. Keratinocytes are the dominant epithelial cells of the epidermis and are connected by tight junctions. Tight junctions play a vital role in maintaining the skin barrier by regulating paracellular permeability and preventing the entry of foreign substances. Together, these structures are essential for epidermal homeostasis, immune defense, and skin integrity [1].

Beyond their structural role, keratinocytes are active participants in innate immunity. They produce antimicrobial peptides, cytokines, and chemokines that regulate inflammation and recruit immune cells to fight against infection. They also express pattern recognition receptors (PRRs) that detect pathogens and initiate immune responses. Their ability to interact with immune cells and modulate skin homeostasis underscores their critical role in barrier function and immune defense.

Eosinophils are granulocytic leukocytes that play crucial roles in immunopathological responses, including allergic reactions and inflammatory conditions. They are characterized by a bilobed nucleus and cytoplasmic granules containing cytotoxic proteins, including major basic protein (MBP) and eosinophil cationic protein (ECP). Eosinophils are primarily regulated by type II cytokines, such as IL-4, IL-5, and IL-13, which promote eosinophil differentiation, activation, and survival.

Eczema is a broad term for a group of inflammatory skin conditions marked by pruritus, erythema, dryness, scaling, lichenification, and recurrent flare-ups. It results from a combination of environmental triggers, genetic predisposition, and immune dysregulation, leading to skin-barrier dysfunction and chronic keratinocyte inflammation. Eczema includes several types, with atopic dermatitis (AD) being the most common form. AD is a chronic inflammatory skin condition. Other forms include contact dermatitis, dyshidrotic eczema, and nummular eczema. Eczema is often associated with type 2 immune responses and may be characterized by increased eosinophil counts and elevated levels of IL-4, IL-13, and IL-31.

Keratinocytes are the primary cell type in the skin and play critical roles in the development of eczema. Impaired keratinocyte function can lead to skin barrier dysfunction, marked by dysregulated tight junctions, increased permeability, and heightened susceptibility to allergens and microbes, thereby contributing to inflammation. Keratinocytes also produce pro-inflammatory cytokines in response to insults, including IL-33, TSLP, and IL-25. Notably, IL-33, TSLP, and IL-25 can recruit eosinophils.

Eosinophils contribute to eczema by disrupting tight junctions, infiltrating inflamed skin, and releasing cytotoxic granules. Cytotoxic components released by eosinophils include, but are not limited to, major basic protein (MBP) and eosinophil cationic protein (ECP), which exacerbate tissue damage and inflammation. Their interaction with Th2 cytokines, such as IL-4 and IL-13, further amplifies allergic inflammation and worsens disease severity.

Psoriasis is a chronic, systemic inflammatory disorder characterized by prominent dermatitis. PSO is a systemic autoimmune disorder characterized by skin barrier dysfunction and inflammation, with elevated levels of the epithelial cytokine TSLP, a feature shared with eczema. Notably, TSLP levels correlate with PSO severity. Elevated pro-inflammatory IL-17 has been observed in patients, but PSO is largely driven by Th17 activation. Patients with PSO, particularly those with severe or rapidly progressive disease, exhibit elevated blood eosinophil counts and eosinophil cationic protein levels.

Emerging evidence supports targeting keratinocytes as a treatment for PSO. Although the disease is immune-driven, keratinocytes actively contribute to inflammation through abnormal hyperproliferation, altered differentiation, and expression of pro-inflammatory mediators [2, 3]. Therapies that normalize keratinocyte function lead to clinical improvement and restoration of epidermal architecture. Moreover, transcriptomic studies show that effective biologic therapies reverse the psoriatic keratinocyte gene signature in parallel with clinical response [4]. Collectively, restoring keratinocyte integrity is central to disease resolution.

LTh(αK) is a genetically detoxified enterotoxoid derived from a human E. coli isolate. It exhibits immunomodulatory activity by interacting with GM1, a glycosphingolipid on the plasma membrane. LTh(αK) consists of two subunits: the A subunit (LTA), a genetically detoxified monomeric protein, and the B subunit (LTB), a pentamer that binds GM1.

The LTh(αK) binds GM1, triggering a series of cellular events that activate innate immune responses. When GM1-expressing cells come into contact with LTh(αK), the pentameric LTBs cross-link GM1 on the plasma membrane, activating internalization signals that promote uptake of the LTh(αK) complex into the endosome, along with the GM1-associated membrane. This process is further directed by endoplasmic reticulum (ER) relocation signals, resulting in translocation and accumulation of GM1 in the ER. As a result, GM1 accumulation leads to calcium efflux from the ER, triggering cellular and mitochondrial stress and innate immune responses.

Innate immune responses are known to involve pro-inflammatory type I interferon activity [5, 6].

Type I interferons (mainly IFN-α and IFN-β) are antiviral cytokines, but their role in inflammation is complex [7]. Rather than being pro-inflammatory or anti-inflammatory, Type I IFNs act as context-dependent regulators whose effects depend on timing, dose, cell type, and disease setting [8]. Type I IFNs are also biphasic. Early, controlled IFNs response may support host defense, whereas prolonged or excessive signaling may suppress immunity or promote pathology [9]. Experimental studies further show that IFN-α can first enhance, then later inhibit, cytokine signaling in T cells [10].

AD17002, which comprises LTh(αK), has been developed into various products, including nasal spray influenza vaccines (NCT03784885) and mucosal immunotherapy for SARS-CoV-2 (NCT05069610, NCT05541510), demonstrating its versatility as a therapeutic agent. However, the role of LTh(αK) in dermatitis remains unexplored.

### Summary of the Invention

Given the central roles of keratinocytes and epithelial innate immune signaling in the pathogenesis of inflammatory skin disorders, LTh(αK) is hypothesized to modulate innate immune pathways by interacting with GM1 on skin resident cells, including but not limited to keratinocytes, tissue resident immune cells, and other epithelial cells, thereby restoring barrier integrity and immune homeostasis. Without being bound by theory, LTh(αK) binding to GM1 may modulate type I interferon, thereby reducing pathogenic type 2 and/or type 17 inflammatory responses, limiting eosinophil recruitment and activation, and promoting normalization of keratinocyte differentiation and tight junction integrity. Accordingly, LTh(αK) may be useful for the prevention, treatment, or amelioration of dermatitis, including, but not limited to, eczema, atopic dermatitis, and PSO, by modulating epithelial-and keratinocyte-mediated innate immune responses.

It is found in the present disclosure that detoxified Escherichia coli heat-labile toxins, especially LTh(αK), are effective in modulating keratinocyte inflammation and reducing the severity of dermatitis. The immunomodulatory potential of LTh(αK) has been demonstrated by its ability to induce innate immune responses and pro-viral activity. It is also found in the present disclosure that detoxified Escherichia coli heat-labile toxins, especially LTh(αK), are effective in treating dermatitis in animal models.

Accordingly, the present disclosure provides a method for treating dermatitis, including but not limited to atopic dermatitis, eczema, or PSO, in subjects by administering a therapeutically effective amount of a composition comprising detoxified Escherichia coli heat-labile toxin (e.g., LTh(αK), such as AD17002). The composition is intended for topical administration. That is, detoxified Escherichia coli heat-labile toxins are applied directly to the affected skin. The detoxified toxins can be formulated into a topical composition and administered as needed or according to standard topical dosages.

Due to their low cost and high efficacy, corticosteroids are commonly used to treat dermatitis. However, because dermatitis is a chronic illness, prolonged corticosteroid treatment has been associated with numerous adverse events, including skin atrophy, striae, delayed wound healing, and skin hyperpigmentation. Biologics, such as antibodies, are alternative medications but are associated with high costs. The present disclosure introduces a biologically based therapy confined to the diseased skin. Moreover, the disclosure offers a safer treatment option for patients with a broad spectrum of inflammatory markers, including Th1 and Th2 cytokines and eosinophils.

The present invention is described in detail in the following sections. Further characterizations, purposes, and advantages of the invention can be found in the detailed descriptions and claims that follow.

### BRIEF DESCRIPTION OF THE FIGURE

The patent or application file contains at least one drawing executed in color. Copies of this patent or patent application publication with color drawings will be provided by the Office upon request and payment of the necessary fee.

FIG. 1 shows the results of LTh(αK) treatment for calcipotriol (MC903)-induced mouse atopic dermatitis (AD) on the ears. Ear thickness, measured daily, reflected the severity of skin inflammation, and the percentage changes following MC903 induction and LTh(αK) treatments were calculated.

FIG. 2 shows the results of LTh(αK) treatment on MC903-induced atopic dermatitis. The erythema, scaling, and lesions on the ears reflect the severity of skin inflammation at the end of the study.

FIG. 3 shows the results of LTh(αK) treatment for Imiquimod cream (IMQ)-induced mouse PSO on the dorsal skin. Dorsal skin thickness, measured daily, reflected the severity of skin inflammation, and the percentage changes following IMQ induction and LTh(αK) treatments were calculated.

FIG. 4 shows the results of LTh(αK) treatment on IMQ-induced PSO over the dorsal skin. The erythema, scaling, and skin thickness (induration) for the dorsal skin reflect the severity of inflammation at the end of the study.

FIG. 5 shows that LTh(αK) treatment attenuates Psoriasis Area and Severity Index (PASI).

### DETAILED DESCRIPTION OF THE INVENTION

Unless otherwise defined herein, scientific and technical terms used in connection with the present disclosure shall have the meaning commonly understood by those of ordinary skill in the art. The meaning and scope of the terms should be clear; however, in the event of any latent ambiguity, the definitions provided herein shall control over any dictionary or extrinsic definition.

As used in this disclosure, the following terms, unless otherwise indicated, shall be understood to have the following meanings:

Throughout this specification, the word “comprise” and its variations, such as “comprises” or “comprising,” are understood to imply the inclusion of a stated integer (or component) or group of integers (or components), but not the exclusion of any other integer (or component) or group of integers (or components).

The singular forms “a,” “an,” and “the” are intended to include the plurals unless the context clearly dictates otherwise. Similarly, plural terms shall include the singular unless the context requires otherwise.

“Treatment” and “treating” and similar terms refer to any approach to achieving a beneficial or desired outcome, including clinical outcomes. For purposes of this disclosure, such outcomes include, but are not limited to, inhibiting or suppressing the onset or progression of a condition, reducing the severity of the condition, minimizing the number and/or intensity of symptoms, improving the quality of life for those affected, reducing the dosage of other medications required, enhancing the effects of another medication a patient is taking, and/or prolonging the survival of patients with the condition.

“Administering” or “administration of” a substance, a compound, or an agent to a subject can be carried out using a variety of methods known to those skilled in the art. For example, a compound or an agent can be administered intranasally. Administration can also be performed, for example, once, a plurality of times, and/or over one or more extended periods. In some aspects, the administration includes both direct administration, including self-administration, and indirect administration, including the act of prescribing a drug. For example, as used herein, a physician who instructs a patient to self-administer a drug, to have the drug administered by another, and/or who provides a patient with a prescription for a drug is administering the drug to the patient.

The term “modulate,” “modulation,” or “modulatory” used herein refers to the regulation of a condition, level, or amount. The regulation may be upregulation or downregulation.

The terms “patient” and “subject” are used interchangeably and refer to either a human or a non-human animal. These terms include mammals such as humans, primates, livestock (including bovines, porcine, etc.), companion animals (e.g., canines, felines), and rodents (e.g., mice and rats).

“Effective amount” refers to the quantity of a therapeutic agent or a pharmaceutically acceptable salt thereof that, based on its efficacy parameters and potential for toxicity, and in light of the knowledge of the practicing specialist, should be effective in a given therapeutic form. As understood in the art, an effective amount can be administered in one or more doses.

In the present disclosure, it was unexpectedly found that a topical composition comprising a GM1-binding or GM1-cross-linking agent, preferably a AB5 toxin, toxoid, or AB5 enterotoxin derivatives, more preferably a genetically detoxified Escherichia coli heat-labile toxin (LT), such as AD17002, is effective in reducing clinical signs of dermatitis. Accordingly, the present invention relates to a method for treating dermatitis that involves administering a therapeutically effective amount of a composition comprising a GM1-binding or GM1-cross-linking agent, preferably a AB5 toxin, toxoid, or AB5 enterotoxin derivatives, more preferably a genetically detoxified Escherichia coli heat-labile toxin (LT), such as AD17002. In one aspect, the composition is for topical administration.

The dermatitis is associated with skin inflammation. In one embodiment, the dermatitis is eczema. In another embodiment, the dermatitis is atopic dermatitis. In another embodiment, the dermatitis is psoriasis.

In one embodiment, the detoxified LT is AD17002 (also called “LTh(αK)”). AD17002 corresponds to LTS61K, as disclosed in US 2008/0102078, which is a genetically detoxified E. coli LT holotoxin with a lysine substitution at position 61 of SEQ ID NO: 5, as disclosed in US 2008/0102078.

The topical administration of the composition may follow general criteria for topical medications. In one embodiment, the composition is administered one or more times. In a preferred embodiment, the composition is administered from weekly to twice daily. In another preferred embodiment, the composition may be administered multiple times daily or weekly. In one embodiment, the composition is administered alone. In another embodiment, the composition is administered in combination with other treatments. In a preferred embodiment, the treatments comprise dermatitis drugs. In a more preferred embodiment, the drugs are for atopic dermatitis. In a more preferred embodiment, the drugs are for PSO. In one embodiment, the composition is administered to the subject upon the occurrence of dermatitis symptoms. In one embodiment, the composition is administered to the subject upon the occurrence of PSO symptoms.

The topical formulation may be formulated with a thickener (or thickeners). In one embodiment, the thickener comprises methylcellulose, such as hydroxypropyl methylcellulose (HPMC). In one embodiment, the thickener is hydrophilic or a hydrophilic base. In one embodiment, the topical formulation further comprises an antimicrobial preservative, including but not limited to polyaminopropyl biguanide (PHMB), nano-silver particles, and ethylhexylglycerin.

Having now generally described the disclosure, the same may be more readily understood through reference to the following examples. The examples are offered for illustrative purposes only and are not intended to limit the scope of the present invention in any way.

### EXAMPLES

### Example 1: Animal Studies for the Attenuation of Dermatitis (Atopic Dermatitis) With Treatment of LTh(αk)

Mice were treated with MC903 (calcipotriol), a vitamin D3 analog widely used to induce AD-like inflammation, to establish an MC903-induced AD animal model. This model recapitulates key features of human AD, including skin barrier dysfunction, immune activation, and Type 2 inflammation.

The mechanisms underlying MC903-induced AD involve skin barrier dysfunction and irritation, TSLP-driven Type 2 inflammation, amplification of IL-33 and IL-25, ILC-2 activation, and recruitment of pro-inflammatory cells, including but not limited to eosinophils and other granulocytes.

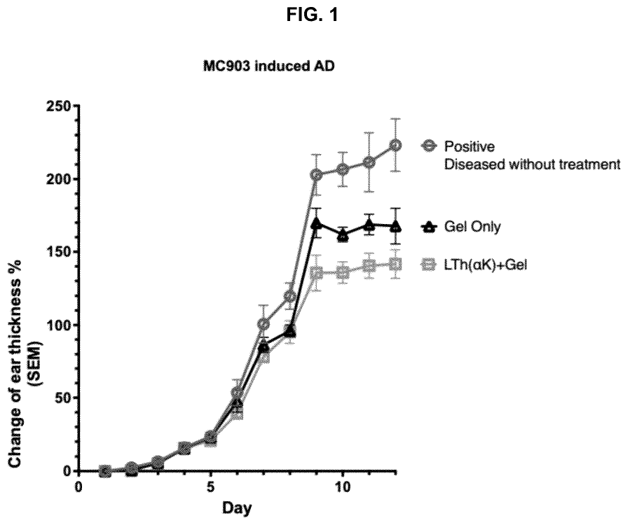

In this study, LTh(αK) was incorporated into a thickener (cellulose-based hydrogel), hydroxypropyl methylcellulose (HPMC), to prolong LTh(αK) hydration after topical application and to extend the duration of contact between LTh(αK) and the target skin. Some antimicrobial preservatives were incorporated into the hydrogel formulation. To induce AD-like symptoms, MC903 was diluted and applied to the mice's ears daily. LTh(αK)−hydrogel treatments were applied to the MC903-treated ears daily, starting when AD symptoms developed. The results are shown in FIG. 1. Mice treated with MC903 alone (red) exhibited the most severe clinical AD symptoms, including inflammation and ear thickness, as indicated by a 223% increase in ear swelling. The hydrogel alone (Gel Only, black) had a moderate effect in reducing the ear thickness (168%). The treatment of LTh(αK)−formulated hydrogel (LTh(αK)+Gel, green) had the greatest therapeutic effect in reducing MC903-induced ear thickness (142%). Notably, the LTh(αK) containing hydrogel significantly reduced MC903-induced AD symptoms.

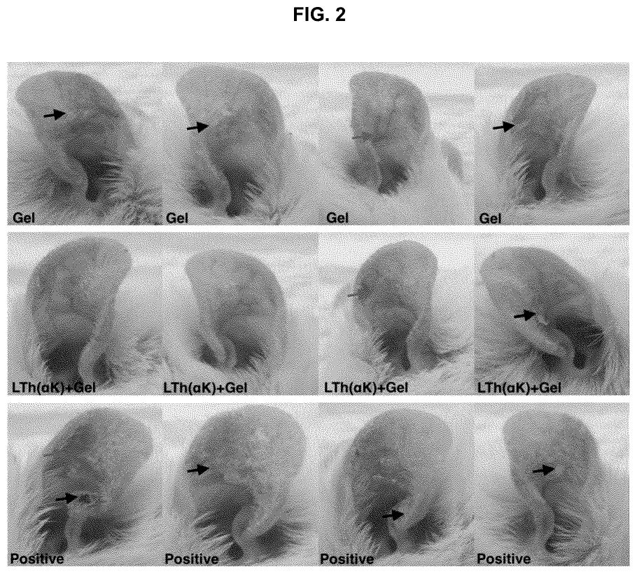

LTh(αK)−incorporated hydrogel reduced other clinical symptoms, such as erythema, scaling, and lesions induced by MC903. As shown in FIG. 2, mice treated with MC903 alone (Positive) exhibited the most severe clinical AD symptoms, as indicated by erythema, scaling, and lesions on the ears (red arrow: erythema; black arrow: scaling). The treatment of hydrogel formulation alone (Gel Only) had a moderate effect in reducing the ear symptoms. The LTh(αK)−hydrogel (LTh(αK)+Gel) had the greatest therapeutic effect in reducing MC903-induced AD symptoms. Notably, the formulation of LTh(αK) resulted in a significant reduction in MC903-induced AD symptoms.

### Example 2: Animal Studies for the Attenuation of Dermatitis (PSO) With Treatment of LTh(αK)

Mice were treated with IMQ (Imiquimod cream), widely used to induce PSO-like dermal inflammation, to establish a PSO animal model. This model recapitulates key features of human PSO, including erythema (redness), scaling (desquamation), skin thickness (induration), immune activation, neutrophil infiltration, and Th17 activation.

The mechanisms underlying topical IMQ application involve binding to TLR7 (Toll-like receptor 7) on dendritic cells, which activates the IL-23/IL-17 axis. Th17 cell activation causes epidermal hyperplasia (thickening), erythema (redness), scaling, and recruitment of pro-inflammatory cells, including but not limited to eosinophils, and other granulocytes—classic signs of PSO.

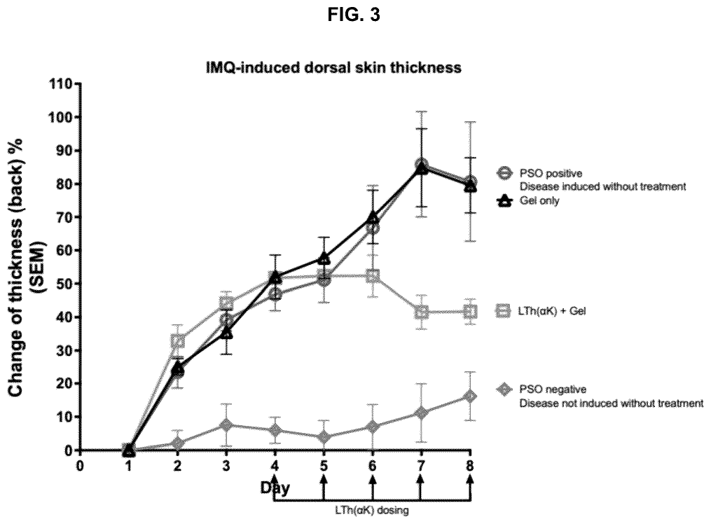

In this study, LTh(αK) was incorporated into a thickener (hydrogel), hydroxypropyl methylcellulose (HPMC), to prolong LTh(αK) hydration after topical application and to extend the duration of contact between LTh(αK) and the target skin. Some antimicrobial preservatives were incorporated into the hydrogel formulation. To induce PSO-like symptoms, daily IMQ (5% cream) was applied to the mice's dorsal skin. LTh(αK)−hydrogel treatments were applied to the IMQ-treated dorsal skin daily starting the day PSO symptoms developed. The results are shown in FIG. 3-5. Mice treated with IMQ alone (Positive, red) exhibited the most severe clinical psoriatic symptoms, including changes in dorsal skin thickness (81%). The hydrogel alone (Gel Only, black) had no effect on IMQ-induced dorsal skin thickness (80%). The LTh(αK)−hydrogel (LTh(αK)+Gel, bright green) had the most therapeutic effect in reducing IMQ-induced dorsal skin thickness 42% (FIG. 3).

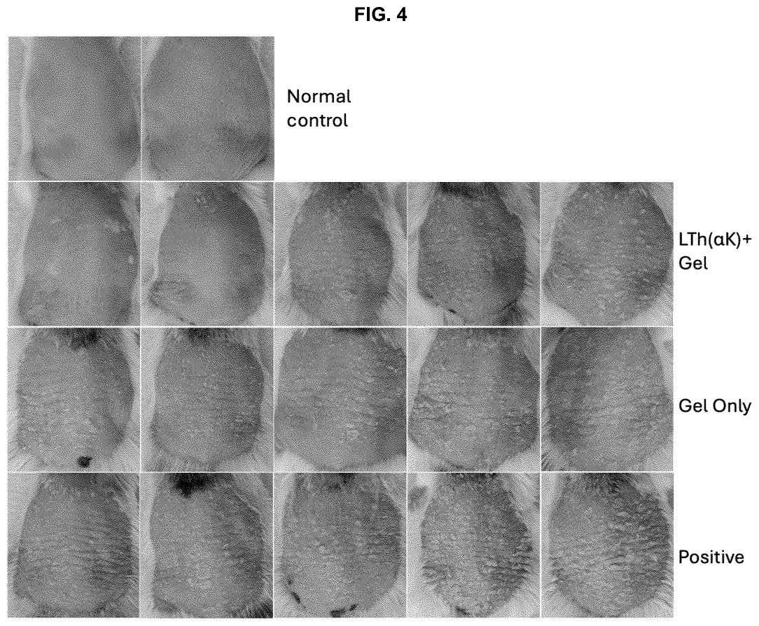

Mice treated with IMQ (Positive) exhibited severe clinical psoriatic symptoms, including but not limited to erythema, scaling, and skin thickening (FIG. 4). The LTh(αK)−hydrogel had a clear therapeutic effect in reducing psoriatic skin symptoms.

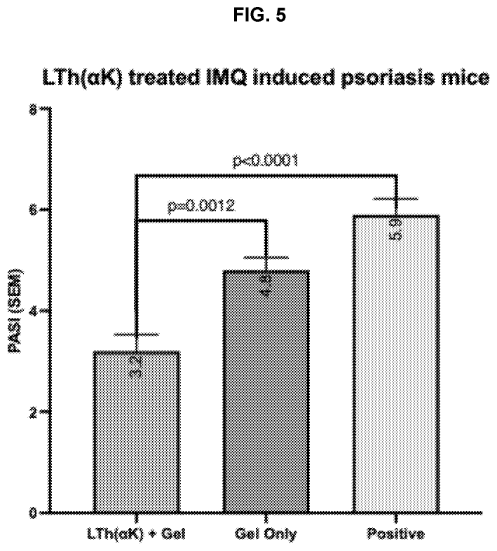

The Psoriasis Area and Severity Index (PASI) is a widely used quantitative tool for assessing the severity of psoriatic skin lesions (FIG. 5). It combines three key clinical features: erythema (redness), scaling (desquamation), and skin thickness (induration). Each feature reflects a distinct pathological aspect of PSO: erythema indicates underlying inflammation and vascular changes, scaling reflects abnormal keratinocyte differentiation and turnover, and thickness denotes epidermal hyperproliferation. In this study, PASI scores were calculated using a 3-point scale to maintain consistency and practicality while still capturing important differences in disease severity. Specifically, each parameter was rated as mild, moderate, or severe, enabling a straightforward yet effective assessment across experimental groups. To reduce subjective bias and improve the reliability of the results, scoring was conducted independently by three evaluators. This approach enhanced objectivity and minimized potential systematic bias. Analysis showed that all three independent scoring datasets revealed consistent trends across the experimental groups. Although there were minor differences in absolute scores and p-values—expected given the subjective nature of visual scoring—each evaluator independently detected statistically significant differences between groups. The overall consistency in trends and statistical outcomes across all three datasets highlights the robustness of the findings and validates the PASI scoring method used. Overall, these data support the conclusion that LTh(αK) is an effective treatment for PSO.

### REFERENCES

1. Heinemann, U. & Schuetz, A. (2019) Structural Features of Tight-Junction Proteins, Int J Mol Sci. 20.
2. Lowes, M. A., Bowcock, A. M. & Krueger, J. G. (2007) Pathogenesis and therapy of psoriasis, Nature. 445, 866-73.
3. Nestle, F. O., Kaplan, D. H. & Barker, J. (2009) Psoriasis, N Engl J Med. 361, 496-509.
4. Shrotri, S., Daamen, A., Kingsmore, K., Bachali, P., Grammer, A. & Lipsky, P. (2025) Transcriptomic Analysis Identifies Disease Severity and Therapeutic Response in Psoriasis, JID Innov. 5, 100333.
5. Pritchard, A. L., Carroll, M. L., Burel, J. G., White, O. J., Phipps, S. & Upham, J. W. (2012) Innate IFNs and plasmacytoid dendritic cells constrain Th2 cytokine responses to rhinovirus: a regulatory mechanism with relevance to asthma, J Immunol. 188, 5898-905.
6. Nakagome, K. & Nagata, M. (2022) Innate Immune Responses by Respiratory Viruses, Including Rhinovirus, During Asthma Exacerbation, Front Immunol. 13, 865973.
7. Anfossi, R., Vivar, R., Ayala, P., Gonzalez-Herrera, F., Espinoza-Perez, C., Osorio, J. M., Roman-Torres, M., Bolivar, S. & Diaz-Araya, G. (2023) Interferon-beta decreases LPS-induced neutrophil recruitment to cardiac fibroblasts, Front Cell Dev Biol. 11, 1122408.
8. Ji, L., Li, T., Chen, H., Yang, Y., Lu, E., Liu, J., Qiao, W. & Chen, H. (2023) The crucial regulatory role of type I interferon in inflammatory diseases, Cell Biosci. 13, 230.
9. McNab, F., Mayer-Barber, K., Sher, A., Wack, A. & O'Garra, A. (2015) Type I interferons in infectious disease, Nat Rev Immunol. 15, 87-103.
10. Eriksen, K. W., Sommer, V. H., Woetmann, A., Rasmussen, A. B., Brender, C., Svejgaard, A., Skov, S., Geisler, C. & Odum, N. (2004) Bi-phasic effect of interferon (IFN)-alpha: IFN-alpha up-and down-regulates interleukin-4 signaling in human T cells, J Biol Chem. 279, 169-76.

What is claimed is:

1. A method for treating a subject with dermatitis, comprising administering to the subject a therapeutically effective amount of a composition comprising a GM1-binding or GM1-cross-linking agent, wherein the composition is administered topically.

2. The method of claim 1, wherein the GM1-binding or GM1-cross-linking agent is a AB5 toxin, toxoid, or AB5 enterotoxin derivatives.

3. The method of claim 2, wherein the AB5 toxin, toxoid, or AB5 enterotoxin derivatives is detoxified Escherichia coli heat-labile toxin (LT).

4. The method of claim 3, wherein the detoxified Escherichia coli heat-labile toxin (LT) is LTh(αK).

5. The method of claim 1, wherein the dermatitis comprises atopic dermatitis.

6. The method of claim 1, wherein the dermatitis comprises eczema.

7. The method of claim 1, wherein the dermatitis comprises psoriasis.

8. The method of claim 1, wherein the composition is administered one or more times.

9. The method of claim 1, wherein the composition is administered alone.

10. The method of claim 1, wherein the composition is administered in combination with other treatments.

11. The method of claim 1, wherein the composition is formulated with a thickener.

12. The method of claim 11, wherein the thickener comprises methylcellulose.

13. The method of claim 12, wherein the methylcellulose is hydroxypropyl methylcellulose.

14. The method of claim 11, wherein the thickener is hydrophilic.

15. The method of claim 11, wherein the composition further comprises an antimicrobial preservative.

16. The method of claim 15, wherein the antimicrobial preservative is a nano-silver particle.

17. The method of claim 15, wherein the antimicrobial preservative is hexamethylene biguanide.

18. The method of claim 15, wherein the antimicrobial preservative is ethylhexylglycerin.
